(19) United States
(12) Patent Application Publication
(10) Pub. No.: US 2009/0169553 A1
(43) Pub. Date: Jul. 2, 2009

(54) Novel Protein Fusion/Tag Technology

(75) Inventor: Duane E. Day, Novi, MI (US)

Correspondence Address: FITZPATRICK CELLA HARPER & SCINTO, 30 ROCKEFELLER PLAZA, NEW YORK, NY, 10112, US

(73) Assignee: Molecular Innovations

(21) Appl. No.: 11/743,791

(22) Filed: May 3, 2007

Related U.S. Application Data
(60) Provisional application No. 60/797,612, filed on May 4, 2006.

(51) Int. Cl. A61K 39/395; C12N 11/02; C12N 15/85; C12N 5/10; C12P 21/00; C12P 21/08; C07K 16/44; C12N 5/16

(52) U.S. Cl. 424/139.1; 435/177; 435/320.1; 435/325; 435/69.7; 435/69.6; 530/387.9

(57) ABSTRACT

The present invention relates to fusion molecules comprising at least one first purification domain and a molecule of interest, wherein the purification domain is selected from the group consisting of a kringle domain and a staphylocoagulase D2 domain. The present invention further relates to methods of purifying a molecule of interest using the fusion molecules of the invention. Also provided is a method of making an antibody using the fusion molecules of the invention, wherein the purification domain is a kringle domain. In addition, the present invention provides for vaccines which have reduced immunoreactivity and which comprise a fusion molecule having a kringle domain and an immunogenic domain.

### CROSS REFERENCE TO RELATED APPLICATIONS

This application claims priority to U.S. Provisional Patent Application No. 60/797,612, filed on May 4, 2006.

### FIELD OF THE INVENTION

The present invention relates to fusion molecules, methods of preparation of such molecules and uses thereof. In particular, the present invention relates to a fusion molecule comprising a purification domain and a molecule of interest. The purification domain may comprise at least one kringle domain and/or domain 2 of staphylocoagulase. These fusion molecules are particularly useful for the purification of proteins and for the production of antibodies, as well as for the production of vaccines.

### BACKGROUND OF THE INVENTION

Kringle domains are triple-disulfide-linked peptide regions of approximately 80 amino acids. The name of this structural protein domain comes from its resemblance to Danish pastries known as kringlers. Kringle domains are found in a varying number of copies in some serine proteases and plasma proteins that evolved from an ancestral gene with a single copy of the kringle domain and that constitute the kringle-serine proteinase superfamily (KSP superfamily of proteins (Gherardi et al, 1997). Kringle domains were observed first in plasminogen (Castellino and McCance, 1997) but analogous structures have been found throughout the blood clotting and fibrinolytic proteins and in a variety of other proteins (Hughes, 2000; Castellino and Beals, 1987). Proteins containing kringle domains include tissue-type plasminogen activator (tPA) and urinary plasminogen activators (uPA) (Gütnzler et al, 1982; Pennica et al, 1983) apolipoprotein A (ApoA), which has as many as 37 repeats of plasminogen K4 (McLean et al, 1987), prothrombin (Walz et al, 1977), coagulation factor XIIa (McMullen and Fujikawa, 1985), tyrosine kinases related to Trk (Wilson et al, 1993), HGF (Lokker et al, 1994), derivatives of HGF such as HGF/NK1, HGF/NK2, HGF/NK4, as well as prothrombin kringle-2 domain (fragment-2), and HGF-like protein (Han et al, 1991). Kringle domains are believed to play a role in binding mediators, such as peptides, other proteins, membranes, or phospholipids (Patthy et al, 1984).

Langer-Safer et al. (J. Biol. Chem., 1991, 266: 3715-3723) replaced the finger and growth factor domain of tPA with K1 from plasminogen. Substitution of these two domains with K1 caused an enhancement in the binding of the tPA chimera to fibrin fragments.

Wu et al. developed a fast-acting clot dissolving agent which includes a clot-targeting domain that is derived from the Kringle 1 domain of human plasminogen which was fused to the C-terminal end of staphylokinase. Wu et al., JBC vol. 278:18199-18206 (2003). This clot-dissolving agent had better clot dissolving activity than the non-fused staphylokinase.

The kringle 5 domain of plasminogen exhibits potent inhibitory effect on endothelial cell proliferation. It can also cause cell cycle arrest and apoptosis of endothelial cell specifically, and shows promise in anti-angiogenic therapy. Zhang et al. (Prep Biochem Biotechnology vol. 35:17-27, 2005) describe a human kringle 5 fusion expressed with a GST (gluthathione-S-transferase) tag. This fusion was purified with glutathione-Sepharose 4B via the GST tag. The GST-kringle 5 fusion protein exhibited some anti-proliferation activity towards bovine capillary endothelial cells. Similarly, a kringle domain of ApoA was fused to a his tag for the purpose of purifying the kringle domain via the his tag. Hrzenjak et al., Protein Engineering vol. 13:661-666 (2000).

Staphylocoagulase (SC) is a protein secreted by the human pathogen, Staphylococcus aureus, that activates human prothrombin (ProT) by inducing a conformational change. Each SC molecule consists of two rod-like helical domains connected at an angle of ˜110°, which include an N-terminal domain (D1; amino acids 1-149) and a C-terminal domain (D2; amino acids 150-282), as shown in FIG. 2. The N-terminal, D1 domain interacts with the 148-loop of thrombin and prothrombin 2 and the south rim of the catalytic site, whereas D2 occupies (pro)exosite I, the fibrinogen (Fbg) recognition exosite.

### SUMMARY OF THE INVENTION

The present invention is based, at least in part, on the observation that kringle domains and staphylocoagulase D2 domains can be used to purify molecules fused thereto. Thus, the present invention relates to fusion molecules comprising at least one first purification domain selected from a kringle domain or a staphylocoagulase D2 domain fused to a molecule of interest, such as a polypeptide, polynucleotide or small molecule. The fusion molecules of the invention may further comprise at least one second purification domain. The present invention further relates to methods for purifying the fusion molecules of the invention. In addition, the present invention relates to methods for making antibodies using the fusion molecules of the invention, and the antibodies made therefrom. Furthermore, the invention relates to vaccines directed to the fusion molecules of the invention.

### DESCRIPTION OF THE DRAWINGS

FIGS. 1A & B depict the structure of human plasminogen and its 5 kringle domains (K1 through K5). FIG. 1A is a simplistic representation and FIG. 1B includes amino acid information and protease cleavage site information, the kringle 1 domain is highlighted in red.

FIG. 2 depicts the structure of staphylocoagulase.

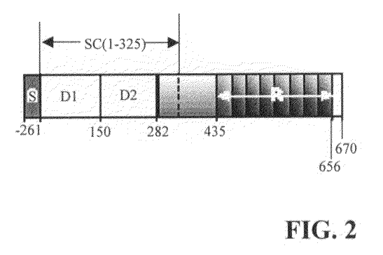

FIG. 3 (A) is an SDS-PAGE gel showing the purity of recombinant human plasminogen and (B) is a graph showing the activation of the purified human plasminogen.

FIG. 4 is an SDS-PAGE gel showing reduced and non-reduced KI-neuroserpin fusion protein.

FIG. 5 is an SDS-PAGE gel showing the purity of K1-neuroserpin fusion protein cleaved with TEV.

FIG. 6 is an SDS-PAGE gel showing purified native neuroserpin in the absence and presence of a molar excess of tPA

FIG. 7 is an SDS-PAGE gel showing the purity of the staphylocoagulase D2 domain.

FIG. 8 is the amino acid sequence of an exemplary human plasminogen (PLGN) Kringle 1-TEV linker of the invention (SEQ ID NO: 1).

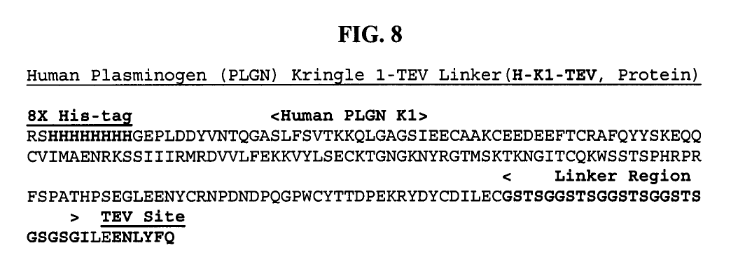

FIG. 9 is the DNA sequence that encodes the amino acid sequence of FIG. 8 (SEQ ID NO:2).

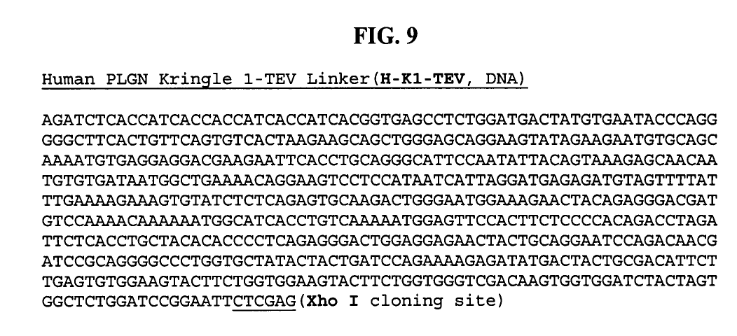

FIG. 10 is the amino acid sequence of an exemplary mouse PLGN Kringle1/Kringle 2 (SEQ ID NO:3).

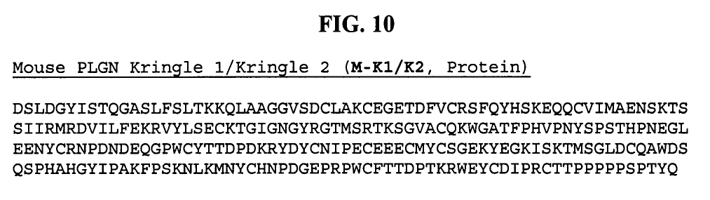

FIG. 11 is the DNA sequence that encodes the amino acid sequence of FIG. 10 (SEQ ID NO.4).

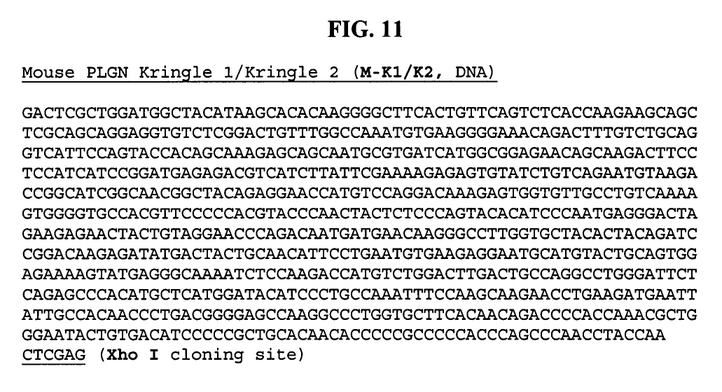

FIG. 12 is the amino acid sequence of an exemplary fusion protein of the invention comprising 8× his tag, human PLGN Kringle1/TEV/Neuroserpin (SEQ ID NO: 5).

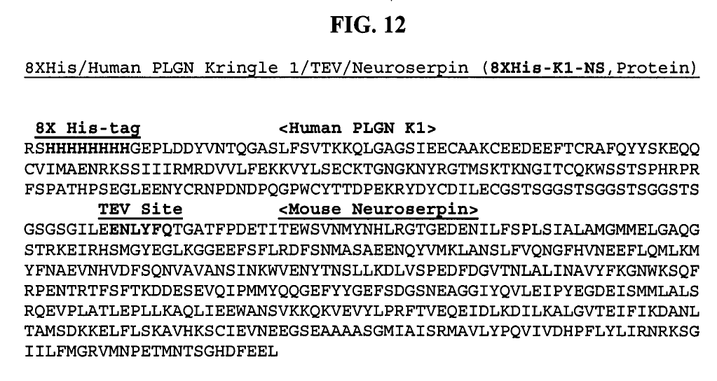

FIG. 13 is the DNA sequence that encodes the amino acid of FIG. 12 (SEQ ID NO:6).

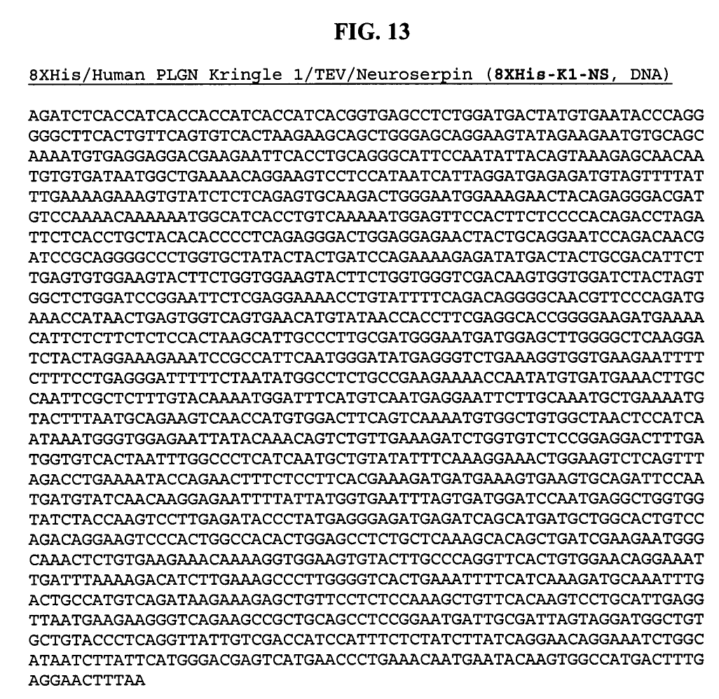

FIG. 14 is the amino acid sequence of an exemplary fusion protein of the invention comprising mouse PLGN Kringle 1 & 2/Human Prostate Specific Antigen (SEQ ID NO:7).

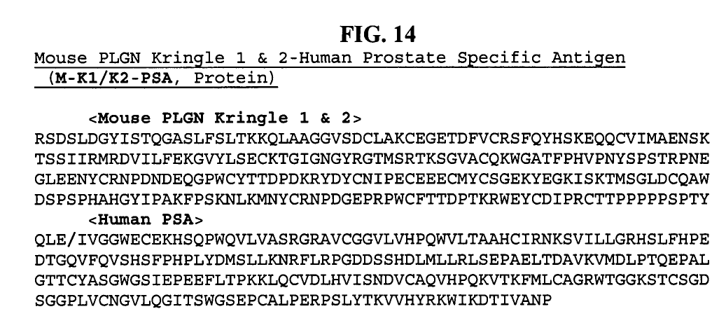

FIG. 15 is the DNA sequence that encodes the amino acid of FIG. 14 (SEQ ID NO:8).

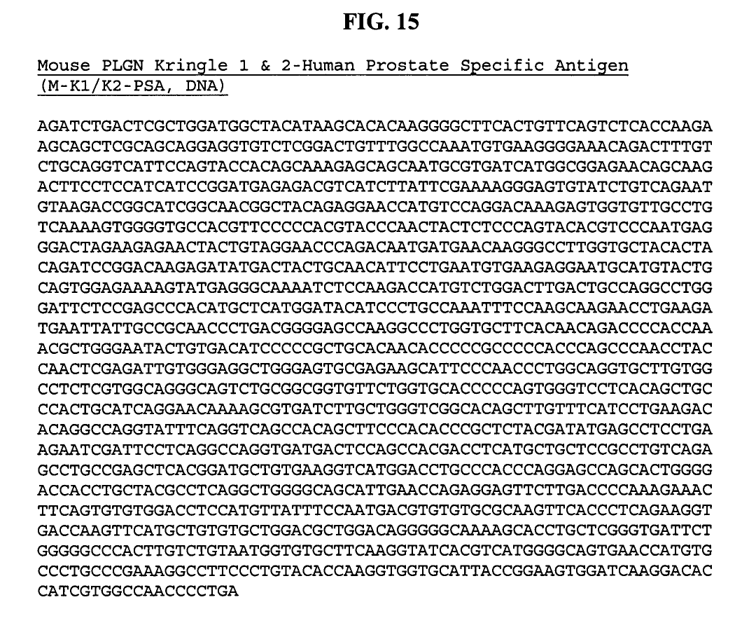

FIG. 16 is an SDS-PAGE gel showing the purity of a recombinant 8× his tag-kringle 1-TEV-rat prorenin fusion protein of the invention.

FIG. 17 is the amino acid sequence of an exemplary fusion protein of the invention comprising 8× his tag, mouse PLGN Kringle 1, TEV & rat prorenin (SEQ ID NO:9).

FIG. 18 is the DNA sequence that encodes the amino acid of FIG. 17 (SEQ ID NO:10).

FIG. 19 is an SDS-PAGE gel showing the purity of a recombinant 8× his tag-kringle 1-TEV-human prorenin fusion protein of the invention.

FIG. 20 is the amino acid sequence of an exemplary fusion protein of the invention comprising 8× his tag, mouse PLGN Kringle 1, TEV & human prorenin (SEQ ID NO:11).

FIG. 21 is the DNA sequence that encodes the amino acid of FIG. 20 (SEQ ID NO:12).

### DETAILED DESCRIPTION OF THE INVENTION

In one embodiment of the invention there is provided fusion molecules comprising at least one first purification domain, wherein the first purification domain is selected from a kringle domain and a staphylocoagulase D2 domain, fused to a molecule of interest.

As used herein, a purification domain is a domain that is capable of binding to a particular moiety and can facilitate the removal of contaminating molecules from the fusion molecules, i.e. facilitate purification of the fusion molecules of the invention. In accordance with the present invention, where the at least one first purification domain is a kringle domain, the domain binds to lysine, lysine analog or fibrin and wherein the purification domain is a D2 domain, the domain binds to prothrombin or thrombin.

As noted above, kringle domains were first observed in plasminogen but analogous domains have been found throughout the blood clotting and fibrinolytic proteins and in a variety of other proteins, tPA, uPA, apolipoprotein A (ApoA), prothrombin, coagulation factor XIIa, tyrosine kinases related to Trk, HGF, derivatives of HGF such as HGF/NK1, HGF/NK2, HGF/NK4, as well as prothrombin kringle-2 domain, and HGF-like protein. Accordingly, the fusion molecules of the present invention may comprise a kringle domain from any source, as well as derivatives, mutations and analogs thereof, as long as the kringle domain has lysine and/or fibrin binding activity.

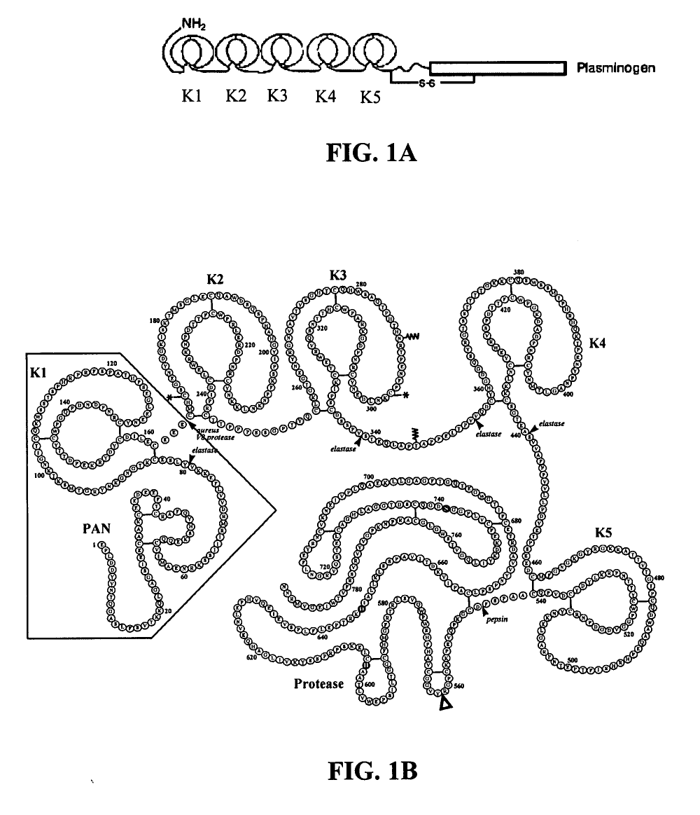

In a particularly preferred embodiment, the fusion molecules of the present invention comprise at least one purification domain that is a kringle domain. Preferably the kringle domain is derived from plasminogen. Plasminogen is a 92 kDa protein present in high concentration in human and animal plasmas. It is converted from an inactive zymogen to the highly digestive fibrinolytic enzyme plasmin via cleavage of a single Arg-Val peptide bond by plasminogen activators, such as tPA or uPA. Plasminogen binds very tightly to its natural substrate target fibrin via its kringle domains. Plasminogen contains five homologous kringle domains, each with a molecular weight of approximately 9 kDa, as shown in FIG. 1.

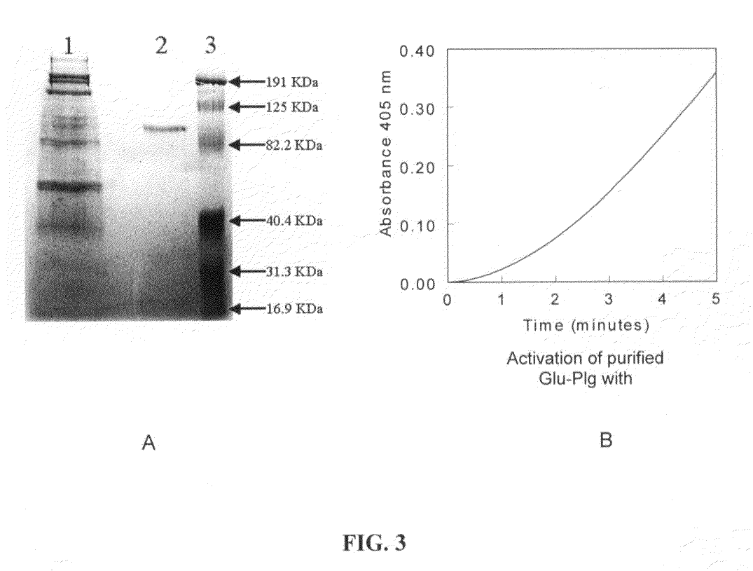

Example 1 and FIG. 3 show that active recombinant human plasminogen can be purified using lysine sepharose. Of the five kringle domains, K1 has been shown to possess the highest fibrin affinity. Christensen & Mølgaard, Biochem J. vol. 285:419-425 (1992).

Clotted fibrin has been shown to be rich in C terminal lysine residues which represent the actual binding sites for the kringle domains. Lucas, M. A., Fretto, L. J. and McKee, P. A. (1983) J. Biol. Chem. 258:4249-56. The present invention is based on part on the lysine binding properties of plasminogen, and in particular the lysine binding properties of the K1 domain of plasminogen. Accordingly, in one embodiment, the fusion molecules of the present invention comprise a purification domain that binds lysine residues. In another embodiment of the invention, the purification domain is the K1 domain of plasminogen and any variants thereof that retain the ability to bind lysine.

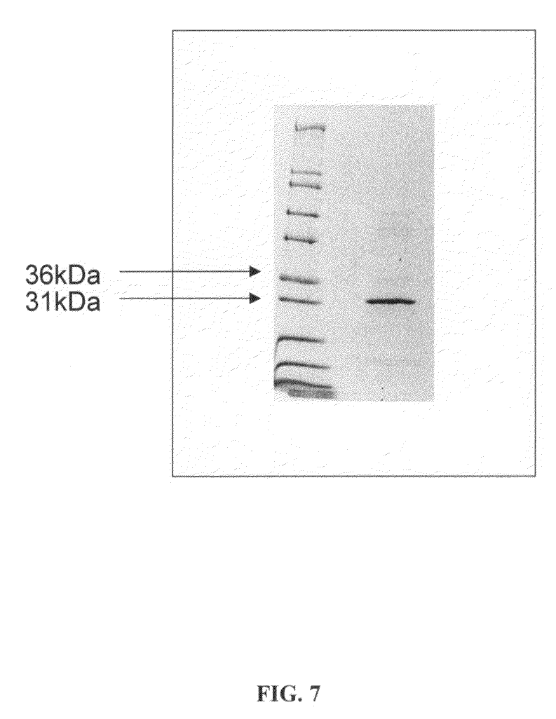

In a further embodiment, the fusion molecules of the present invention comprise a first purification domain that is the D2 domain of staphylocoagulase (SC). SC binds with very high affinity to an exosite present in prothrombin via domain 2. Panizzi et al., J. Biol. Chem., Vol. 281:1169-1178 (2006). Friedrich et al. demonstrated that the D2 domain binds very tightly to prothrombin but lacks the functional activity necessary to induce proteinase activity. Accordingly, the D2 domain, or variant thereof, that retains its ability to bind to prothrombin. Example 3 and FIG. 7 show that recombinant D2 can be purified with a prothrombin-coupled resin.

As noted above, the fusion proteins of the present invention may further comprise at least one second purification domain, which in preferred embodiments is selected from a His tag and a HAT tag, although any known purification domain may be included in the fusion molecules of the invention to further facilitate purification of the fusion molecules. Other purification domains known to the skilled artisan include those selected from GST, MBP, FLAG, CBP, CYD, HPC and Strep II, all commonly known as “tags”. The HAT tag system uses a similar concept to histidine tag technology, where the ability of histidine to bind to metal ions facilitates purification of a histidine tagged molecule. For the HAT tag system, which is described by Jiang et al., Journal of Virology, Vol. 78: 8994-9006 (2004), several histidines are included in a tag but are separated by other amino acids, which has the effect of making fusion proteins incorporating the tag theoretically more soluble.

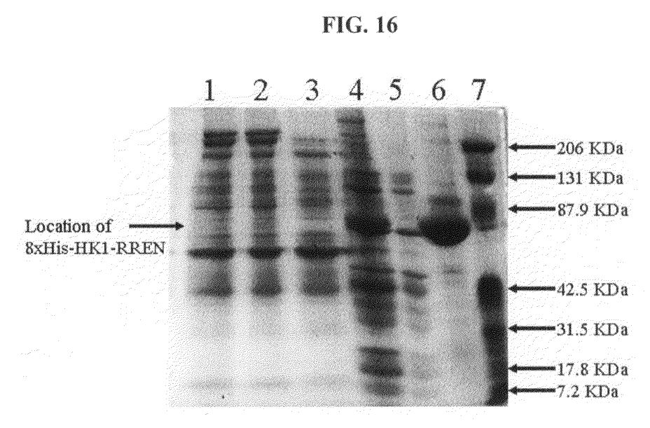

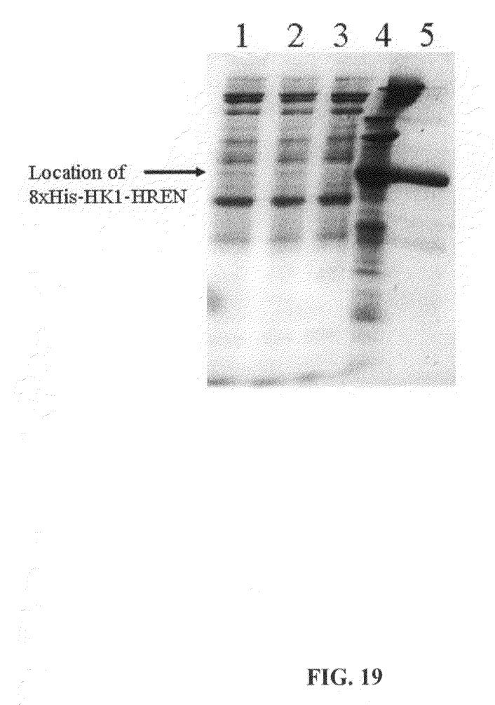

Example 4 and FIG. 16 show that recombinant rat prorenin fusion protein comprising the K1 domain (first purification domain) and a 8×His tag (second purification domain) can be purified using a metal chelating matrix via the 8×His tag and lysine Sepharose® via the K1 domain. Example 5 and FIG. 19 correspondingly show that recombinant human prorenin fusion protein comprising the K1 domain (first purification domain) and a 8×His tag (second purification domain) may be purified using a metal chelating matrix (Talon® charged with cobalt) via the 8×His tag and lysine Sepharose®& via the K1 domain.

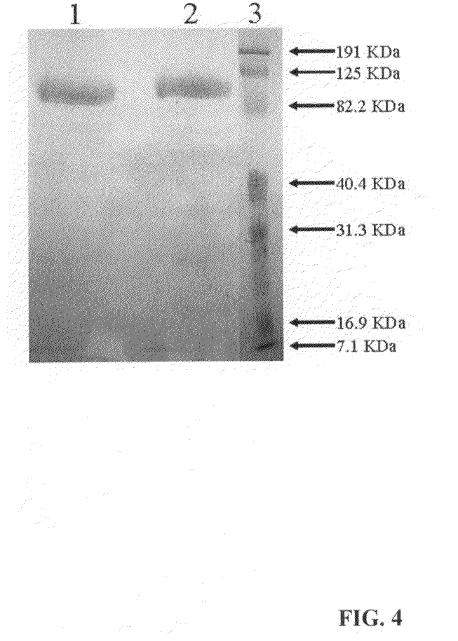

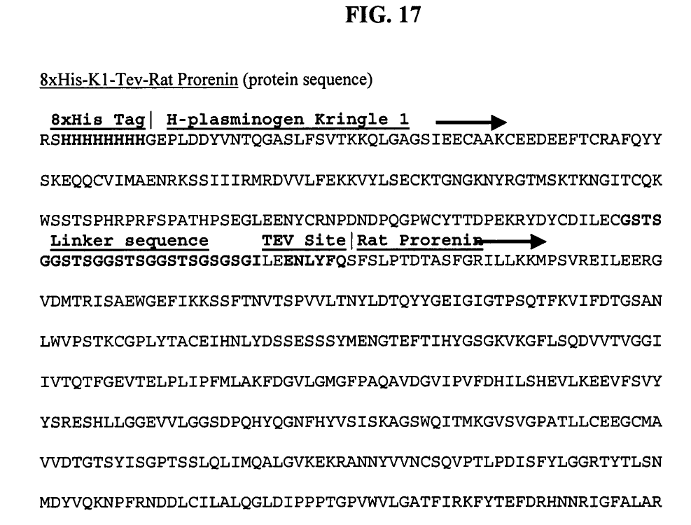

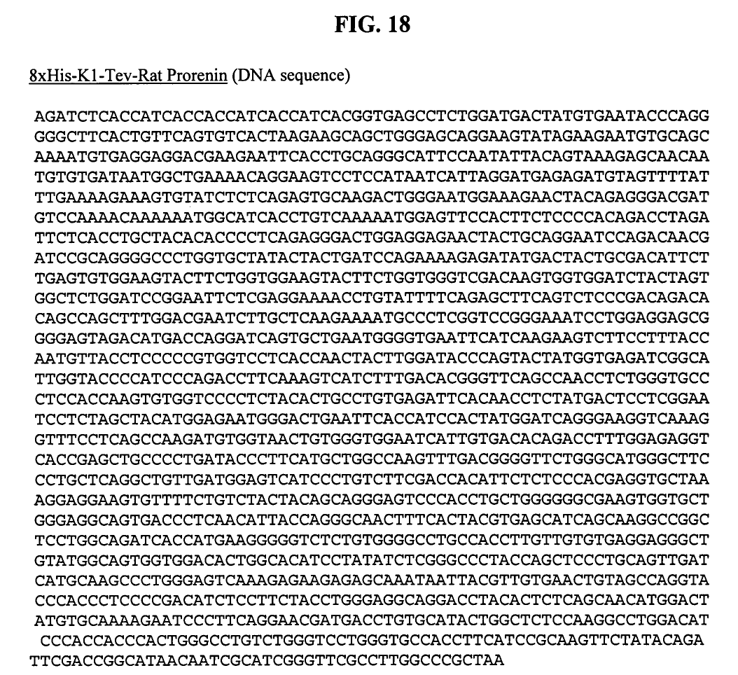

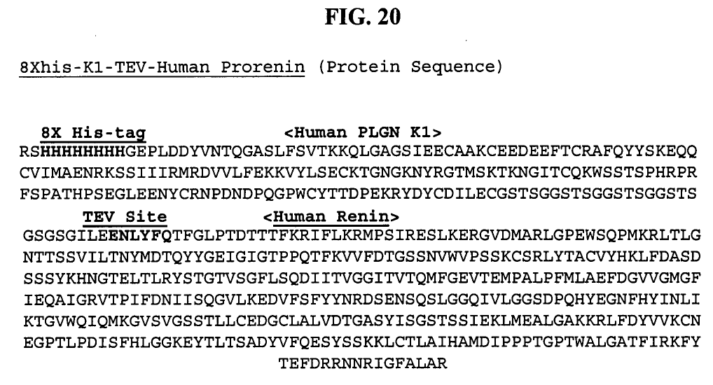

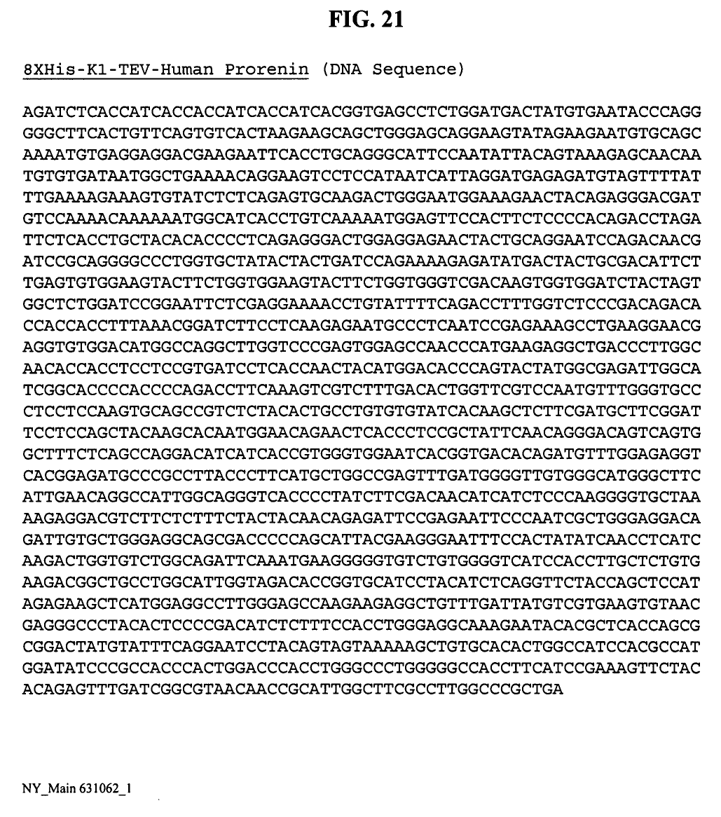

As indicated, the fusion molecules of the present invention further comprise a molecule of interest linked or fused to the at least one purification domain of the invention. Such molecules include polypeptides, polynucleotides and/or small molecules, such as therapeutic compounds. In one embodiment, the molecule of interest is a mouse neuroserpin, as further described in Example 2 below, as well as FIGS. 4, 5 and 6. In another embodiment, the molecule of interest is a rat prorenin, as further described in Example 4 below, as well as FIGS. 16, 17 and 18. In a further embodiment, the molecule of interest is a human prorenin, as further described in Example 5 below, as well as FIGS. 19, 20 and 21.

In addition, the fusion molecules of the present invention may further comprise a cleavage domain that may be included between the purification domain and the molecule of interest, wherein the cleavage domain includes a cleavage sequence. Such a sequence enables the removal of the purification domain from the molecule of interest. Many such sequences are known in the art. Any well known cleavage domain may be included in the fusion proteins of the invention. For example, a protease recognition sequence may be included that is specifically recognized by a protease, such as enterokinase (recognizes the amino acid sequence DDDDK-X (SEQ ID NO:13)), Factor Xa (recognizes the amino acid sequence I-E[D]-G-R (SEQ ID NO:14)) and thrombin. In a particularly preferred embodiment, a recognition sequence for tobacco etch virus (TEV) may be included in the fusion protein of the invention. TEV recognizes the amino acid sequence ENLYFQS (SEQ ID NO:15). Cleavage occurs between the glutamine and serine (serine may be replaced by nearly any amino acid, with the exception of proline). Because serine is not required, the cleavage site can be engineered such that the amino acid adjacent to glutamine is part of the fusion protein to be released, thus resulting in a purified protein with a wild-type sequence. In another preferred embodiment, a recognition sequence for renin may be included in the fusion protein of the invention. It is believed that renin recognizes the amino acid sequence Ile-His-Pro-Phe-His-Leu-Val-Ile-His-Asn (SEQ ID NO:16) and that renin cleaves at the Leu-Val bond leaving four amino acids on the N-terminus of the target polypeptide that it cleaves. In a further preferred embodiment, a recognition sequence for urokinase (uPA) may be included n the fusion protein of the invention. Examples of such recognition sequences are found in a publication by Ke et al., The Journal of Biological Chemistry, vol. 272, no. 33, pp 20456-62 (1997) (see, e.g., Table III, sequences VI and VII). An example of a fusion molecule according to the invention comprising such a cleavage site is further described in Examples 2, 4 and 5 below.

The fusion molecules of the present invention may be in the form of polynucleotides, polypeptides, and small molecules or any combination thereof. The fusion molecule may be a polynucleotide that encodes a fusion protein of the invention, comprising a purification domain and a polypeptide of interest. The present invention is also directed to expression vectors comprising polynucleotide sequences. The present invention further provides for host cells that comprise the vectors of the present invention in which the fusion molecules may be expressed, as further described in the Examples below.

In addition, the present invention provides for methods of purifying the fusion molecules of the present invention from the host cells in which they are expressed. In one embodiment, where the fusion molecule comprises at least one first purification domain which includes at least one kringle domain, or variant thereof, the method of purifying the fusion molecule comprises a first purification step of administering the fusion molecule, which may be in a cell lysate, to a matrix which comprises immobilized lysine, lysine analogs, such as epsilon amino caproic acid (6-amino hexanoic acid), and fibrin or derivatives thereof. In this regard, the kringle-comprising fusion molecule will bind to the matrix allowing contaminants to be removed. The fusion molecule may be removed from the matrix by the addition of free lysine or epsilon amino caproic acid (EACA), which will compete for the binding of the fusion protein to the immobilized lysine. In a particularly preferred embodiment, the matrix is a lysine Sepharose® resin, as further described in Examples 3, 4 and 5 below.

In another embodiment, where the fusion molecule comprises at least one first purification domain which includes at least one D2 domain, or variant thereof, the method of purifying the fusion molecule comprises a first purification domain purification step of administering the fusion molecule, which may be in a cell lysate, to a matrix which comprises immobilized prothrombin. In this regard, the D2-comprising fusion molecule will bind to the matrix allowing contaminants to be removed. The fusion molecule may be removed from the matrix by the addition of thiocyanate, which is a chaotropic salt that disrupts protein/protein interactions. In a particularly preferred embodiment, the matrix is Affi-Gel 10 (Bio-rad, Hercules, Calif.) coupled to prothrombin, as further described in Example 2 below.

In a further embodiment, where the fusion molecules of the invention comprise a second purification domain which is a 8×His domain, the method of purifying the fusion molecule includes a second purification domain purification step. The second purification domain purification step may occur before or after the first purification domain purification step. The His tag of the fusion molecules will bind to the metal chelating matrix allowing contaminants to be removed. Qiagen (Hilden, Germany) manufactures and sells a metal chelate resin which can be charged with a divalent metal, such as cobalt, nickel, zinc, copper and manganese, and also provides detailed instructions for purification using the resin. Valen Biotech, Inc. (Atlanta, Ga.) manufactures and sells pre-packed metal chelating resins, called IMAC resins which may also be charged with a divalent metal, such as cobalt, nickel, zinc, copper and manganese. In addition, Amersham Pharmacia Biotech, Inc. (Piscataway, N.J.) manufactures and sells metal chelate resins, such as HiTrap™ Chelating HP. Further, BD Biosciences Clontech (Palo Alto, Calif.) manufactures and sells Talon resins which were used in the present invention (see Examples 4 and 5).

The purification methods may further comprise isolating the molecule of interest from the purification domain by including a cleavage domain in the fusion molecule of the invention between the molecule of interest and the purification domain. The purification methods would thus further include a cleavage step wherein the purification domain is removed from the molecule of interest and the molecule of interest is recovered.

The present invention also provides methods for preparing antibodies and antibody cell lines prepared thereby. The generation of monoclonal and polyclonal antibodies is well known in the art. The methods and antibody cell lines of the present invention allow for the production of antibodies in the absence of contaminating antibodies.

The method for preparing the antibodies of the invention utilizes the kringle fusion molecules of the invention. In this regard, a fusion protein may be made that comprises a kringle domain from the species in which the antibodies will be prepared. For example, the kringle domain may be from mouse if mice will be used to generate the antibodies or rabbit if rabbits will be used. The antigen of interest is fused to this species-specific kringle domain. When the fusion protein is administered to the animal in which antibodies will be prepared, the animal will not mount a response or may mount a weak response to the kringle domain because it is native to the animal. However, the animal will mount a response to the antigen of interest. Accordingly, antibodies can be produced with fewer non-specific antibody contaminants. The fusion protein may be purified prior to its administration to the animal via the kringle domain. In addition, the antibodies can be purified from the animal, or a hybridoma cell line of the invention, via the fusion protein, ensuring the purification of only the antibodies that bind to the antigen of interest.

The present invention further provides for the polyclonal and monoclonal antibodies produced by the methods of the present invention, as well as antibody fragments (e.g. Fab, and F(ab′)₂) and recombinantly-produced binding partners which specifically bind to the fusion proteins of the invention.

An immortal cell line that produces a monoclonal antibody of the present invention is also part of the present invention. In a specific embodiment of this immortal cell line, the monoclonal antibody is prepared against the fusion proteins of the invention.

The purified antibodies may be incorporated into various pharmaceutical compositions, and these compositions may be administered by any means known in the art to achieve the intended purpose. Amounts and regimens for the administration of these compositions can be determined readily by those with ordinary skill in the clinical art of treating any of the particular diseases.

In a further embodiment, the present invention provides for a vaccine comprising the fusion molecules of the present invention. The vaccine of the present invention may comprise a fusion molecule of the invention which comprises a kringle domain and an immunogenic domain, wherein the immunogenic domain is capable of raising an immune response in a host that boosts immunity to a microorganism, such as a bacterium or a virus. The kringle domain is preferably a kringle domain of the host and is therefore non-immunogenic or weakly immunogenic. For example, where the host is human, the kringle domain may be the K1 domain of human plasminogen. In one embodiment, the immunogenic domain may be a portion of the DENV envelope protein. For example, a fusion protein may be created using the K1 domain of the invention, wherein the fusion protein is part of the viral coat of a vaccine. The k1 domain may allow for the purification of the vaccine. Further, the K1 domain may be added to a pegylated virus for the purpose of facilitating purification of the pegylated virus.

The following nonlimiting examples serve to further illustrate the present invention.

### EXAMPLES

### Example 1

### Amplification, Cloning, Expression and Purification of Human Plasminogen

The entire sequence of human Glu plasminogen was cloned and expressed in a drosophila (DS2) cell expression system. The DS2 cell line was previously described by Schneider in 1972. Schneider, J Embryol Exp Morphol. 27:353-65 (1972). Drosophila cell culture is desirable because drosophila cells exhibit mammalian-like post-translational modification, including core glycosylation patterns similar to mammalian cells. This provides an advantage over bacterial expression systems which lack such modifications, such as proper disulfide bond formation and glycosylation and which may result in the production of denatured or insoluble proteins. Other cells may be used in accordance with the invention such as bacterial and mammalian cells.

The cDNA encoding human plasminogen was amplified by thermocycling using oligonucleotide primers designed to generate the mature human plasminogen from human liver cDNA (Boichain, cat. #C1234149). The primer sequences were as follows: 5′ ATT ACT CGG GGA GCC TCT GGA TGA CTA TGT G (SEQ ID NO:17); and 3′ ATT ACT CGA GTT AAT TAT TTC TCA TCA CTC CCT G (SEQ ID NO:18). The primers were designed to generate a flush, in frame junction with the BiP signal sequence in pMT-BiP-B vector. The gene was cloned according to standard techniques, transfected into competent E. coli, and amplified plasmids were screened by restriction digestion using standard protocols. The positive clones were confirmed by DNA sequencing.

The completed plasmid was transfected into drosophila S2 cells using Cellfectin (Invitrogen). The plasmid and the Blasticidin resistance gene vector (pCO-BLAST) were co-precipitated in 0.3M sodium acetate and 2.5 volumes of 95% ethanol, centrifuged and sterilized by washing with 70% ethanol and resuspended in serum free medium (D-SFM) without antibiotics and Cellfectin was added dropwise to the DNA with agitation. The DNA mixture was then combined with approximately 5×10⁶ pelleted DS2 cells previously washed with D-SFM lacking antibiotics. The cells were added to a T25 tissue culture flask and incubated at room temperature for four hours, followed by the addition of 3 ml D-SFM with antibiotics. Cells were incubated for 48 hours at room temperature, pelleted by centrifugation, resuspended into complete D-SFM containing 25 μg/ml Balsticidin to select for transfected cells. The transfected cells were grown in D-SFM with antibiotics and induced by the addition of 500 μM copper sulfate, followed by incubation at room temperature with agitation (115 rpm) for 3-6 days. The cultures were then clarified by centrifugation and 1000×g (Sorvall RC-B %, GSA rotor). The conditioned medium containing the expressed recombinant plasminogen was decanted from the pellets.

To determine whether recombinant plasminogen could be purified using a lysine matrix, the conditioned media was concentrated by ammonium sulfate (55%) precipitation and pelleted by centrifugation at 7000 g for 20 minutes. The resultant pellet was resuspended in binding buffer (0.1 M HEPES, 0.1 M NaCl; pH 7.4) and dialyzed extensively against the same buffer to remove lysine present in the culture media. Lysine Sepharose™ 4B resin was used as the lysine matrix. The resin was in the form of a column which was first washed with 2-3 bed volumes of binding buffer prior and approximately 10 bed volumes of media sample was applied to the column. The column was then washed extensively until the readout reached baseline at 280 nm absorbance. Non-specifically bound contaminants were eluted from the column in 0.5 M sodium chloride. The plasminogen was eluted with 0.2 M epsilon amino caproic acid (EACA) in the binding buffer. The EACA was removed by dialysis.

FIG. 3A shows the plasminogen purified via lysine Sepharose, row 1 is the cell culture media, row 2 is the purified recombinant human plasminogen and row 3 is standard. Approximately 20 mg/L of cell culture was obtained. To determine whether the recovered plasminogen was correctly folded and functional an activation assay was used. 25 μl of purified plasminogen was added to a cuvette containing 1 ml of a 200 μM solution of chromogenic plasmin substrate (D-VLK, Molecular Innovations, Inc.). 1 μl of 0.1 mg/ml uPA was added and the absorbance at 405 nm was monitored continuously as a function of time. Conversion of the plasminogen to active plasmin was evidenced by the characteristic hyperbolic trace shown in FIG. 3B. This shows that the entire molecule was properly folded as evidenced by its binding to lysine sepharose through its N-terminal K1 domain and the generation of a functional proteinase domain which resides in the C-terminal portion of the plasminogen molecule.

### Example 2

### Purification of K1-tev-Neuroserpin Fusion Protein

Neuroserpin is a protein that is expressed throughout the nervous system and inhibits the serine protease tissue plasminogen activator (tPA). It is believed to be involved in neural development, neural growth, synaptic plasticity, memory, stroke, epilepsy and Alzheimer's disease.

In accordance with the invention, a polynucleotide was made which expresses a fusion protein comprising the K1 domain of human plasminogen, the TEV cleavage site and mouse neuroserpin, as described above for human plasminogen. Clones were isolated and used to transfect DS2 cells as described above. The cells were grown and induced and the supernatant was collected and purified using lysine Sepharose®. Solid ammonium sulfate was added to the DS2 cell media. 0.4 grams were added per ml of media. The sample containing dissolved ammonium sulfate was chilled at 4° C. for one hour and the pellet collected by centrifugation. The protein pellet was dissolved in a TBS buffer and dialyzed against the same. The dialyzed sample was applied to a column of immobilized lysine and washed with TBS buffer. The column was developed with a linear gradient consisting of TBS in the proximal chamber and 10 mM EACA in the distal chamber. The mouse neuroserpin/K1 fusion eluted at an EACA concentration of approximately 3 mM. The sample was collected and concentrated to approximately 2-3 mg/ml and dialyzed against the TBS buffer to remove the εACA.

FIG. 4 shows the fusion protein under non-reducing and reducing conditions. Mouse neuroserpin is known to contain a single cysteine residue which in theory could result in dimer formation. The non-reducing gel shows no evidence of a higher molecular weight species suggesting that the cysteine may be inaccessible. Interestingly, the fusion protein is biologically active for neuroserpin as determined in a chromogenic assay with human tPA. SDS Page shows the purified fusion protein to be greater than 99% pure as evidenced by the lack of contaminating insect cell proteins.

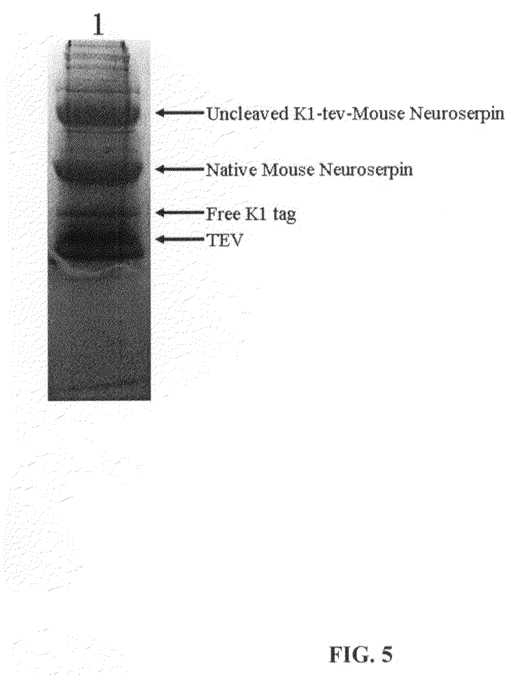

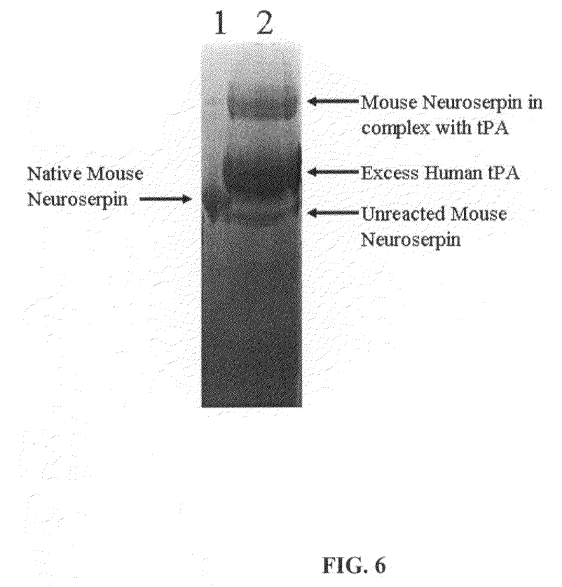

FIG. 5 shows the purified fusion protein which has been cleaved with TEV releasing the native wild-type mouse neuroserpin protein. Mouse neuroserpin was separated from both TEV, unreacted fusion protein and free K1 by hydrophobic affinity chromatography on Phenyl Sepharose®. Briefly, solid ammonium sulfate was added to the reaction mixture (0.1 grams per ml sample) and applied to a phenyl Sepharose® column equilibrated with TBS buffer containing 30% saturated ammonium sulfate. A linear gradient was used to develop the column. The proximal chamber contained the TBS with 30% saturated ammonium sulfate and the distal chamber contained the TBS buffer with no ammonium sulfate. The free mouse neuroserpin eluted early in the gradient whereas all other components separated out well into the elution. K1 appeared to bind very tightly to the resin and both the free K1 as well as unreacted K1/fusion elute near the limit of the gradient. FIG. 6 demonstrates that the TEV cleaved purified native sequence recombinant neuroserpin has tPA binding activity as evidenced by SDS stable complex formation.

### Example 3

### Purification of the Staphylocoagulase D2 Domain Produced in E. Coli

The D2 domain of staphylocoagulase was previously cloned into an E. Coli expression system. The ability of D2 to be expressed and subsequently purified from media using immobilized human prothrombin was demonstrated. SC D2-TEV (having the D2 domain and the tobacco etch virus cleavage site) was expressed from Rosetta (DE3) plysS cells and induced with 20 g/l lactose for 12-16 hours at 37° C. The cells were harvested by centrifugation and resuspended in 50 mM HEPES, 125 mM NaCl, 1 mg/ml polyethylene glycol (PEG) 8000, 1 mM EDTA, 0.02% sodium azide, pH 7.4. The cells were then lysed by 3 cycles of sonication (˜45 seconds/cycle) on ice, centrifuged to clarify lysates and dialyzed into 50 mM HEPES buffer described above.

Immobilized prothrombin was prepared by coupling (4-5 mg/ml resin coupled) to Affi-Gel 10 (Bio-Rad, Hercules, Calif.) according to the manufacturers instructions. The cell sample was applied to the resin that was pre-washed with the HEPES buffer. The protein was eluted with the HEPES buffer containing 3 M sodium thiocyante (NaSCN). NaSCN is a chaotropic salt that disrupts protein/protein interactions. The eluted sample was dialyzed into 50 mM HEPES, 125 mM NaCl, pH 7.4. The purity of the protein was assessed by 4-15% SDS PAGE gel as shown in FIG. 7. The D2 domain migrates as a homogeneous band at approximately the predicted molecular weight of 26 kDa (row 2 of FIG. 7).

### Example 4

### Purification of 8× his-K1-tev-rat Prorenin Fusion Protein

Renin is a hormone secreted by cells of the kidney which interacts with a plasma protein substrate to produce a decapeptide that is converted to angiotensin II by a converting hormone. Angiotensin II effects vasoconstriction, the secretion of aldosterone by the adrenal cortex, and retention of sodium by the kidney. Renin plays a role in both normal cardiovascular homeostasis and in renovascular hypertension. It also appears that renin plays an important role in maintaining blood pressure and that it is responsible for the initial phases of renovascular hypertension.

Prorenin is a precursor to renin and for many years was considered to be inactive with no function of its own. Chronic stimulation of the renal-angiotensin system usually increases renal prorenin-renin conversion, thereby decreasing the relative amount of prorenin in the circulation. However, there are some reports that prorenin has a function in certain diabetic subjects who have microalbuminaria and in pregnant women, both groups having increased prorenin levels. It is believed that prorenin has renin-like activity when it is bound to its receptor. Thus renin and prorenin are considered potential targets for drugs to treat cardiovascular and renal diseases.

In accordance with the invention, a polynucleotide was made which expresses a fusion protein comprising eight histidines, the K1 domain of human plasminogen, the TEV cleavage site and rat prorenin, as described above for human plasminogen. Clones were isolated and used to transfect DS2 cells as described above. The cells were grown and induced with copper sulfate and the supernatant was collected. Chelex® 100 was added to the supernatant to remove copper sulfate (used to induce expression) from the sample. Then the sample was applied to a Talon® metal chelating column charged with cobalt and washed with TBS buffer. The column was eluted with 200 mM imidizole. The eluate, comprising partially purified rat prorenin fusion protein was then applied to a column of immobilized lysine (lysine Sepharaose®) for further purification. The lysine Sepharose® column was then washed with TBS buffer and developed with a linear gradient consisting of TBS in the proximal chamber and 10 mM EACA in the distal chamber. The rat prorenin/K1-8×His fusion eluted at an εACA concentration of approximately 2 mM. The sample was collected and concentrated to approximately 1 mg/ml and dialyzed against the TBS buffer to remove the εACA.

FIG. 16 is a 10% SDS-PAGE gel showing the purification of the 8×his-k1-rat prorenin fusion protein. Lane 1 is the cell media which includes the fusion protein. Lane 2 shows the media after it has been treated with Chelex®. Lane 3 is the flow through from the metal chelating Talon® matrix. Lane 4 is the eluate from the metal chelating matrix (which was eluted in the presence of 200 mM imidizole). Lane 5 is the flow through from the lysine Sepharose matrix. Lane 6 is the eluate from the Lysine Sepharose matrix and represents the purified 8×his-human k1-rat prorenin fusion. Lane seven is prestained markers.

### Example 5

### Purification of 8× his-K1-tev-human Prorenin Fusion Protein

In accordance with the invention, a polynucleotide was made which expresses a fusion protein comprising eight histidines, the K1 domain of human plasminogen, the TEV cleavage site and human prorenin, as described above for human plasminogen. Clones were isolated and used to transfect DS2 cells as described above. The cells were grown and induced with copper sulfate and the supernatant was collected and purified in the same manner as was the rat prorenin fusion protein of Example 4 above.

FIG. 19 is a 10% SDS-PAGE gel showing the purification of the 8×his-k1-human prorenin fusion protein. Lane 1 is the cell media which includes the fusion protein. Lane 2 shows the media after it has been treated with Chelex®. Lane 3 is the flow through from the metal chelating Talon® matrix. Lane 4 is the eluate from the metal chelating matrix (which was eluted in the presence of 200 mM imidizole). Lane 5 is the flow through from the lysine Sepharose matrix.

What is claimed is:

1. A fusion molecule comprising at least one first purification domain and a molecule of interest, wherein the first purification domain is selected from the group consisting of a kringle domain and a staphylocoagulase D2 domain, and variants thereof.

2. The fusion molecule of claim 1 wherein the kringle domain is capable of binding to lysine.

3. The fusion molecule of claim 2, wherein the kringle domain is selected from a kringle domain of plasminogen.

4. The fusion molecule of claim 3, wherein the plasminogen is human plasminogen.

5. The fusion molecule of claim 4, wherein the kringle domain is the K1 domain of human plasminogen.

6. The fusion molecule of claim 1, wherein the staphylocoagulase D2 domain is capable of binding to prothrombin.

7. The fusion molecule of claim 1 wherein the molecule is selected from the group consisting of a polynucleotide and a polypeptide.

8. The fusion molecule of claim 1 wherein the molecule of interest is a polypeptide, polynucleotide, or a therapeutic agent.

9. The fusion molecule of claim 1 further comprising a protease cleavage site that is located in between the purification domain and the molecule of interest.

10. The fusion molecule of claim 9 wherein the cleavage site is selected from the group consisting of a tobacco etch virus (TEV) cleavage site, an enterokinase cleavage site, a factor Xa cleavage site, a thrombin cleavage site, a renin cleavage site and a uPA cleavage site.

11. The fusion molecule of claim 1, further comprising at least one second purification domain.

12. The fusion molecule of claim 11, wherein the at least one second purification domain is selected from the group consisting of a His tag and a HAT tag.

13. The fusion molecule of claim 12, wherein the at least one second purification domain is a His tag.

14. A vector comprising the fusion molecule of claim 1, wherein the fusion molecule is a polynucleotide.

15. A host cell comprising the vector of claim 14.

16. A method of purifying a fusion molecule comprising (a) generating a fusion molecule according to claim 1, wherein the first purification domain is a polypeptide; (b) applying the polypeptide to a matrix that binds to the first purification domain; (c) and recovering the purified fusion molecule from the matrix.

17. The method of claim 16, wherein the purification domain is a kringle domain and the matrix is selected from the group consisting of a lysine matrix, a lysine analog matrix and a fibrin matrix.

18. The method of claim 16, wherein the purification domain is staphylocoagulase D2 and the matrix is selected from the group consisting of a prothrombin matrix and a thrombin matrix.

19. The method of claim 16, wherein the fusion molecule further comprises a second purification domain.

20. The method of claim 16, wherein the fusion molecule further comprises a protease cleavage site between the purification domain and the molecule of interest.

21. The method of claim 20, further comprising cleaving the purification domain from the molecule of interest and recovering the molecule of interest.

22. A method of making an antibody comprising: (a) administering a fusion molecule according to claim 1 to an animal to generate antibodies therein, wherein the purification domain of the fusion molecule is a kringle domain of the same species of the animal and wherein the purification domain is a polypeptide.

23. The method according to claim 22, further comprising recovering the antibody from the animal.

24. The method according to claim 22, wherein the animal is selected from the group consisting of mouse, rabbit and sheep.

25. The method according to claim 22, wherein the antibody is selected from the group consisting of a monoclonal antibody and a polyclonal antibody.

26. An antibody made by the method of claim 22.

27. A hybridoma cell line expressing the antibody of claim 26.

28. A vaccine comprising a fusion molecule which comprises a kringle domain and an immunogenic domain.


SEQUENCE LISTING (18 sequences)

SEQ ID NO: 1 - protein, 206 aa, Artificial
  Fusion protein
RSHHHHHHHHGEPLDDYVNTQGASLFSVTKKQLGAGSIEECAAKCEEDEEFTCRAFQYYSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECGSTSGGSTSGGSTSGGSTSGSGSGILEENLYFQ

SEQ ID NO: 2 - DNA, 600 bp, Artificial
  Fusion nucleic acid
agatctcaccatcaccaccatcaccatcacggtgagcctctggatgactatgtgaatacccagggggcttcactgttcagtgtcactaagaagcagctgggagcaggaagtatagaagaatgtgcagcaaaatgtgaggaggacgaagaattcacctgcagggcattccaatattacagtaaagagcaacaatgtgtgataatggctgaaaacaggaagtcctccataatcattaggatgagagatgtagttttatttgaaaagaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtggaagtacttctggtggaagtacttctggtgggtcgacaagtggtggatctactagtggctctggatccggaattctcgag

SEQ ID NO: 3 - protein, 255 aa, Artificial
  Fusion protein
DSLDGYISTQGASLFSLTKKQLAAGGVSDCLAKCEGETDFVCRSFQYHSKEQQCVIMAENSKTSSIIRMRDVILFEKRVYLSECKTGIGNGYRGTMSRTKSGVACQKWGATFPHVPNYSPSTHPNEGLEENYCRNPDNDEQGPWCYTTDPDKRYDYCNIPECEEECMYCSGEKYEGKISKTMSGLDCQAWDSQSPHAHGYIPAKFPSKNLKMNYCHNPDGEPRPWCFTTDPTKRWEYCDIPRCTTPPPPPSPTYQ

SEQ ID NO: 4 - DNA, 771 bp, Artificial
  Fusion nucleic acid
gactcgctggatggctacataagcacacaaggggcttcactgttcagtctcaccaagaagcagctcgcagcaggaggtgtctcggactgtttggccaaatgtgaaggggaaacagactttgtctgcaggtcattccagtaccacagcaaagagcagcaatgcgtgatcatggcggagaacagcaagacttcctccatcatccggatgagagacgtcatcttattcgaaaagagagtgtatctgtcagaatgtaagaccggcatcggcaacggctacagaggaaccatgtccaggacaaagagtggtgttgcctgtcaaaagtggggtgccacgttcccccacgtacccaactactctcccagtacacatcccaatgagggactagaagagaactactgtaggaacccagacaatgatgaacaagggccttggtgctacactacagatccggacaagagatatgactactgcaacattcctgaatgtgaagaggaatgcatgtactgcagtggagaaaagtatgagggcaaaatctccaagaccatgtctggacttgactgccaggcctgggattctcagagcccacatgctcatggatacatccctgccaaatttccaagcaagaacctgaagatgaattattgccacaaccctgacggggagccaaggccctggtgcttcacaacagaccccaccaaacgctgggaatactgtgacatcccccgctgcacaacacccccgcccccacccagcccaacctaccaactcgag

SEQ ID NO: 5 - protein, 600 aa, Artificial
  Fusion protein
RSHHHHHHHHGEPLDDYVNTQGASLFSVTKKQLGAGSIEECAAKCEEDEEFTCRAFQYYSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECGSTSGGSTSGGSTSGGSTSGSGSGILEENLYFQTGATFPDETITEWSVNMYNHLRGTGEDENILFSPLSIALAMGMMELGAQGSTRKEIRHSMGYEGLKGGEEFSFLRDFSNMASAEENQYVMKLANSLFVQNGFHVNEEFLQMLKMYFNAEVNHVDFSQNVAVANSINKWVENYTNSLLKDLVSPEDFDGVTNLALINAVYFKGNWKSQFRPENTRTFSFTKDDESEVQIPMMYQQGEFYYGEFSDGSNEAGGIYQVLEIPYEGDEISMMLALSRQEVPLATLEPLLKAQLIEEWANSVKKQKVEVYLPRFTVEQEIDLKDILKALGVTEIFIKDANLTAMSDKKELFLSKAVHKSCIEVNEEGSEAAAASGMIAISRMAVLYPQVIVDHPFLYLIRNRKSGIILFMGRVMNPETMNTSGHDFEEL

SEQ ID NO: 6 - DNA, 1803 bp, Artificial
  Fusion nucleic acid
agatctcaccatcaccaccatcaccatcacggtgagcctctggatgactatgtgaatacccagggggcttcactgttcagtgtcactaagaagcagctgggagcaggaagtatagaagaatgtgcagcaaaatgtgaggaggacgaagaattcacctgcagggcattccaatattacagtaaagagcaacaatgtgtgataatggctgaaaacaggaagtcctccataatcattaggatgagagatgtagttttatttgaaaagaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtggaagtacttctggtggaagtacttctggtgggtcgacaagtggtggatctactagtggctctggatccggaattctcgaggaaaacctgtattttcagacaggggcaacgttcccagatgaaaccataactgagtggtcagtgaacatgtataaccaccttcgaggcaccggggaagatgaaaacattctcttctctccactaagcattgcccttgcgatgggaatgatggagcttggggctcaaggatctactaggaaagaaatccgccattcaatgggatatgagggtctgaaaggtggtgaagaattttctttcctgagggatttttctaatatggcctctgccgaagaaaaccaatatgtgatgaaacttgccaattcgctctttgtacaaaatggatttcatgtcaatgaggaattcttgcaaatgctgaaaatgtactttaatgcagaagtcaaccatgtggacttcagtcaaaatgtggctgtggctaactccatcaataaatgggtggagaattatacaaacagtctgttgaaagatctggtgtctccggaggactttgatggtgtcactaatttggccctcatcaatgctgtatatttcaaaggaaactggaagtctcagtttagacctgaaaataccagaactttctccttcacgaaagatgatgaaagtgaagtgcagattccaatgatgtatcaacaaggagaattttattatggtgaatttagtgatggatccaatgaggctggtggtatctaccaagtccttgagataccctatgagggagatgagatcagcatgatgctggcactgtccagacaggaagtcccactggccacactggagcctctgctcaaagcacagctgatcgaagaatgggcaaactctgtgaagaaacaaaaggtggaagtgtacttgcccaggttcactgtggaacaggaaattgatttaaaagacatcttgaaagcccttggggtcactgaaattttcatcaaagatgcaaatttgactgccatgtcagataagaaagagctgttcctctccaaagctgttcacaagtcctgcattgaggttaatgaagaagggtcagaagccgctgcagcctccggaatgattgcgattagtaggatggctgtgctgtaccctcaggttattgtcgaccatccatttctctatcttatcaggaacaggaaatctggcataatcttattcatgggacgagtcatgaaccctgaaacaatgaatacaagtggccatgactttgaggaactttaa

SEQ ID NO: 7 - protein, 496 aa, Artificial
  Fusion protein
RSDSLDGYISTQGASLFSLTKKQLAAGGVSDCLAKCEGETDFVCRSFQYHSKEQQCVIMAENSKTSSIIRMRDVILFEKGVYLSECKTGIGNGYRGTMSRTKSGVACQKWGATFPHVPNYSPSTRPNEGLEENYCRNPDNDEQGPWCYTTDPDKRYDYCNIPECEEECMYCSGEKYEGKISKTMSGLDCQAWDSPSPHAHGYIPAKFPSKNLKMNYCRNPDGEPRPWCFTTDPTKRWEYCDIPRCTTPPPPPSPTYQLEIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 8 - DNA, 1491 bp, Artificial
  Fusion nucleic acid
agatctgactcgctggatggctacataagcacacaaggggcttcactgttcagtctcaccaagaagcagctcgcagcaggaggtgtctcggactgtttggccaaatgtgaaggggaaacagactttgtctgcaggtcattccagtaccacagcaaagagcagcaatgcgtgatcatggcggagaacagcaagacttcctccatcatccggatgagagacgtcatcttattcgaaaagggagtgtatctgtcagaatgtaagaccggcatcggcaacggctacagaggaaccatgtccaggacaaagagtggtgttgcctgtcaaaagtggggtgccacgttcccccacgtacccaactactctcccagtacacgtcccaatgagggactagaagagaactactgtaggaacccagacaatgatgaacaagggccttggtgctacactacagatccggacaagagatatgactactgcaacattcctgaatgtgaagaggaatgcatgtactgcagtggagaaaagtatgagggcaaaatctccaagaccatgtctggacttgactgccaggcctgggattctccgagcccacatgctcatggatacatccctgccaaatttccaagcaagaacctgaagatgaattattgccgcaaccctgacggggagccaaggccctggtgcttcacaacagaccccaccaaacgctgggaatactgtgacatcccccgctgcacaacacccccgcccccacccagcccaacctaccaactcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcttgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctga

SEQ ID NO: 9 - protein, 590 aa, Artificial
  Fusion protein
RSHHHHHHHHGEPLDDYVNTQGASLFSVTKKQLGAGSIEECAAKCEEDEEFTCRAFQYYSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECGSTSGGSTSGGSTSGGSTSGSGSGILEENLYFQSFSLPTDTASFGRILLKKMPSVREILEERGVDMTRISAEWGEFIKKSSFTNVTSPVVLTNYLDTQYYGEIGIGTPSQTFKVIFDTGSANLWVPSTKCGPLYTACEIHNLYDSSESSSYMENGTEFTIHYGSGKVKGFLSQDVVTVGGIIVTQTFGEVTELPLIPFMLAKFDGVLGMGFPAQAVDGVIPVFDHILSHEVLKEEVFSVYYSRESHLLGGEVVLGGSDPQHYQGNFHYVSISKAGSWQITMKGVSVGPATLLCEEGCMAVVDTGTSYISGPTSSLQLIMQALGVKEKRANNYVVNCSQVPTLPDISFYLGGRTYTLSNMDYVQKNPFRNDDLCILALQGLDIPPPTGPVWVLGATFIRKFYTEFDRHNNRIGFALAR

SEQ ID NO: 10 - DNA, 1772 bp, Artificial
  Fusion nucleic acid
agatctcaccatcaccaccatcaccatcacggtgagcctctggatgactatgtgaatacccagggggcttcactgttcagtgtcactaagaagcagctgggagcaggaagtatagaagaatgtgcagcaaaatgtgaggaggacgaagaattcacctgcagggcattccaatattacagtaaagagcaacaatgtgtgataatggctgaaaacaggaagtcctccataatcattaggatgagagatgtagttttatttgaaaagaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtggaagtacttctggtggaagtacttctggtgggtcgacaagtggtggatctactagtggctctggatccggaattctcgaggaaaacctgtattttcagagcttcagtctcccgacagacacagccagctttggacgaatcttgctcaagaaaatgccctcggtccgggaaatcctggaggagcggggagtagacatgaccaggatcagtgctgaatggggtgaattcatcaagaagtcttcctttaccaatgttacctcccccgtggtcctcaccaactacttggatacccagtactatggtgagatcggcattggtaccccatcccagaccttcaaagtcatctttgacacgggttcagccaacctctgggtgccctccaccaagtgtggtcccctctacactgcctgtgagattcacaacctctatgactcctcggaatcctctagctacatggagaatgggactgaattcaccatccactatggatcagggaaggtcaaaggtttcctcagccaagatgtggtaactgtgggtggaatcattgtgacacagacctttggagaggtcaccgagctgcccctgatacccttcatgctggccaagtttgacggggttctgggcatgggcttccctgctcaggctgttgatggagtcatccctgtcttcgaccacattctctcccacgaggtgctaaaggaggaagtgttttctgtctactacagcagggagtcccacctgctggggggcgaagtggtgctgggaggcagtgaccctcaacattaccagggcaactttcactacgtgagcatcagcaaggccggctcctggcagatcaccatgaagggggtctctgtggggcctgccaccttgttgtgtgaggagggctgtatggcagtggtggacactggcacatcctatatctcgggccctaccagctccctgcagttgatcatgcaagccctgggagtcaaagagaagagagcaaataattacgttgtgaactgtagccaggtacccaccctccccgacatctccttctacctgggaggcaggacctacactctcagcaacatggactatgtgcaaaagaatcccttcaggaacgatgacctgtgcatactggctctccaaggcctggacatcccaccacccactgggcctgtctgggtcctgggtgccaccttcatccgcaagttctatacagattcgaccggcataacaatcgcatcgggttcgccttggcccgctaa

SEQ ID NO: 11 - protein, 592 aa, Artificial
  Fusion protein
RSHHHHHHHHGEPLDDYVNTQGASLFSVTKKQLGAGSIEECAAKCEEDEEFTCRAFQYYSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECGSTSGGSTSGGSTSGGSTSGSGSGILEENLYFQTFGLPTDTTTFKRIFLKRMPSIRESLKERGVDMARLGPEWSQPMKRLTLGNTTSSVILTNYMDTQYYGEIGIGTPPQTFKVVFDTGSSNVWVPSSKCSRLYTACVYHKLFDASDSSSYKHNGTELTLRYSTGTVSGFLSQDIITVGGITVTQMFGEVTEMPALPFMLAEFDGVVGMGFIEQAIGRVTPIFDNIISQGVLKEDVFSFYYNRDSENSQSLGGQIVLGGSDPQHYEGNFHYINLIKTGVWQIQMKGVSVGSSTLLCEDGCLALVDTGASYISGSTSSIEKLMEALGAKKRLFDYVVKCNEGPTLPDISFHLGGKEYTLTSADYVFQESYSSKKLCTLAIHAMDIPPPTGPTWALGATFIRKFYTEFDRRNNRIGFALAR

SEQ ID NO: 12 - DNA, 1779 bp, Artificial
  Fusion nucleic acid
agatctcaccatcaccaccatcaccatcacggtgagcctctggatgactatgtgaatacccagggggcttcactgttcagtgtcactaagaagcagctgggagcaggaagtatagaagaatgtgcagcaaaatgtgaggaggacgaagaattcacctgcagggcattccaatattacagtaaagagcaacaatgtgtgataatggctgaaaacaggaagtcctccataatcattaggatgagagatgtagttttatttgaaaagaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtggaagtacttctggtggaagtacttctggtgggtcgacaagtggtggatctactagtggctctggatccggaattctcgaggaaaacctgtattttcagacctttggtctcccgacagacaccaccacctttaaacggatcttcctcaagagaatgccctcaatccgagaaagcctgaaggaacgaggtgtggacatggccaggcttggtcccgagtggagccaacccatgaagaggctgacccttggcaacaccacctcctccgtgatcctcaccaactacatggacacccagtactatggcgagattggcatcggcaccccaccccagaccttcaaagtcgtctttgacactggttcgtccaatgtttgggtgccctcctccaagtgcagccgtctctacactgcctgtgtgtatcacaagctcttcgatgcttcggattcctccagctacaagcacaatggaacagaactcaccctccgctattcaacagggacagtcagtggctttctcagccaggacatcatcaccgtgggtggaatcacggtgacacagatgtttggagaggtcacggagatgcccgccttacccttcatgctggccgagtttgatggggttgtgggcatgggcttcattgaacaggccattggcagggtcacccctatcttcgacaacatcatctcccaaggggtgctaaaagaggacgtcttctctttctactacaacagagattccgagaattcccaatcgctgggaggacagattgtgctgggaggcagcgacccccagcattacgaagggaatttccactatatcaacctcatcaagactggtgtctggcagattcaaatgaagggggtgtctgtggggtcatccaccttgctctgtgaagacggctgcctggcattggtagacaccggtgcatcctacatctcaggttctaccagctccatagagaagctcatggaggccttgggagccaagaagaggctgtttgattatgtcgtgaagtgtaacgagggccctacactccccgacatctctttccacctgggaggcaaagaatacacgctcaccagcgcggactatgtatttcaggaatcctacagtagtaaaaagctgtgcacactggccatccacgccatggatatcccgccacccactggacccacctgggccctgggggccaccttcatccgaaagttctacacagagtttgatcggcgtaacaaccgcattggcttcgccttggcccgctga

SEQ ID NO: 13 - protein, 6 aa, Artificial
  recognition sequence in any organism for enterokinase
DDDDKX

SEQ ID NO: 14 - protein, 4 aa, Artificial
  recognition sequence in any organism for Factor Xa
IXGR

SEQ ID NO: 15 - protein, 7 aa, Artificial
  recognition sequence in any organism for tobacco etch virus protease
ENLYFQS

SEQ ID NO: 16 - protein, 10 aa, Artificial
  recognition sequence in any organism of renin
IHPFHLVIHN

SEQ ID NO: 17 - DNA, 31 bp, Homo Sapiens
attactcggggagcctctggatgactatgtg

SEQ ID NO: 18 - DNA, 34 bp, Homo Sapiens
attactcgagttaattatttctcatcactccctg